(19) United States
(12) Patent Application Publication
(10) Pub. No.: US 2022/0062335 A1
(43) Pub. Date: Mar. 3, 2022

(54) ANTIMICROBIAL COMPOSITION WITH PROCOAGULANT, IMMUNOMODULATORY, AND TISSUE REGENERATIVE PROPERTIES

(71) Applicant: Hyprotek, Inc., Spokane, WA (US)

(72) Inventor: Patrick O. Tennican, Spokane, WA (US)

(21) Appl. No.: 17/423,440

(22) PCT Filed: Jan. 27, 2020
(86) PCT No.: PCT/US20/15265

§ 371 Date: Jul. 15, 2021

Related U.S. Application Data
(60) Provisional application No. 62/797,901, filed on Jan. 28, 2019.
(60) Provisional application No. 62/819,485, filed on Mar. 15, 2019.

(51) Int. Cl. A61K 33/42; A61K 31/722; A61K 31/045; A61K 33/40; A61K 38/39; A61K 31/728; A61K 33/06

(52) U.S. Cl. CPC A61K 33/42; A61K 31/722; A61K 31/045; A61K 33/06; A61K 38/39; A61K 31/728; A61K 33/40

(57) ABSTRACT

The present disclosure relates to a novel composition of antimicrobial and procoagulant agents and methods of using the composition for inhibiting and treating microbial growth at wound sites, inhibiting and treating microbial infections (e.g., bacterial, fungal, viral, parasitic) at wound sites, modulating excessive local and endemic immune reaction, inhibiting microbial infections in biofilms at wound sites, downregulating initial microbial infection at wound sites, preventing cell lysis associated with microbial biofilms at wound sites, inactivating bacterial toxins at wound sites, skin and mucous membranes, preventing the loss of injured extremities, preventing septic shock, reducing superficial bleeding from wounds, promoting tissue regeneration, and promoting the healing of wounds.

### TECHNICAL FIELD

The present disclosure relates to novel mixtures of antimicrobial agents and procoagulant agents, and methods of using the mixtures for treating wounds to promote healing and tissue regeneration, and prevent or reduce infection, toxins, disease, and/or loss of blood and plasma.

### BACKGROUND

Treating mass casualties, such as occur in natural disasters or combat, has been traditionally challenging both because of the potential severity of the injuries and the number of victims. When casualties occur in low-resource environments, the challenge is exacerbated by the distance and difficult transportation to medical facilities. As a result of patients not being able to get timely and adequate care, there is a substantial risk that wounds will become infected. Depending on the severity of the injury, fatalities may result from loss of blood, absorption of microbial toxins, invasive infection, septicemia, hemolytic uremic syndrome, or septic toxic shock and respiratory failure.

For example, a 2018 study found that although the conflicts in Iraq and Afghanistan have had a battlefield mortality rate lower than in past conflicts, one-third of combat casualties in those conflicts develop skin, soft tissue, or bone infections during their initial hospitalization. Weintrob, A, et al. Early Infections Complicating the Care of Combat Casualties from Iraq and Afghanistan. Surgical Infections. 2018. doi:10.1089/274.2017.240. Use of forward surgical assets, tourniquets, rapid evacuation, and improved body armor resulted in a greater percentage of combat casualties surviving their initial injuries compared with past conflicts, but this reduced mortality rate is coupled with major challenges in subsequent care because of massive blood loss, soft tissue and bone injuries, and extensive wound contamination requiring frequent surgeries. Weintrob.

Frequency and severity of wounds incurred during combat has increased due to improvised explosive devices (IEDs). Additionally, civilian casualties during warfare may also be widespread and occur in limited-resource environments.

Conventionally, methods of hemostasis such as pressure from application of tourniquets or compression at the site of the wound may reduce bleeding to some extent and for some amount of time. However, these techniques do not have antimicrobial effects and so while bleeding may be controlled, infection is not. Additionally, tourniquets require periodic releasing and replacing and so may be impractical when there is a shortage of medical professionals to monitor the patient.

In addition to the challenges of trying to stop the bleeding, wound infections may result from inadequate antiseptic preparations of the skin or cross-infection from fluids of other patients or from contaminated hands of caregivers. During a microbial infection, various cellular stress responses, for example, cytokines, also are also triggered, leading to tissue inflammation and immune cell activation. Chronic inflammation from infection may promote the development of and/or sustain pathways that underlie downstream disorders such as cancer.

Being able to quickly, inexpensively, effectively, and safely reduce both bleeding and infection in early-stage wounds will result in fewer lives lost, less severe permanent injuries, less multidrug resistant infection, and cost savings from reducing the length of treatments, hospitalizations, and rehabilitation.

### SUMMARY

This Summary is provided to introduce a selection of concepts in a simplified form that are further described below in the Detailed Description. This Summary is not intended to identify key or essential features of the claimed subject matter, nor is it intended to be used as an aid in determining the scope of the claimed subject matter.

This disclosure describes a composition for application on wounds and surfaces that has both antimicrobial and hemostatic properties, facilitates or moderates host immune response, and promotes tissue regeneration and remodeling. The composition, when applied to a wound, may prevent or treat infection, and stop the loss of blood and plasma.

The present disclosure relates to the discovery of a novel combination of ingredients that collectively are effective as both an antimicrobial agent and a procoagulant (blood clotting) agent. Accordingly, the present disclosure describes compositions and methods of using the composition for, for example, (1) inhibiting and treating microbial growth, microbial infections, and conditions that may be regulated or associated with microbial infections, (2) stopping or reducing blood loss, and (3) faster wound healing as compared to an antimicrobial agent alone. In an embodiment, the composition can incorporate small molecules for biofilm penetration and broad spectrum, rapid microbial killing, and large molecules for rapid hemostasis and prevention of infection.

In an embodiment, the composition includes an antimicrobial agent in addition to a procoagulant agent. The antimicrobial agent as described herein may comprise (a) water; (b) a low molecular weight alcohol; (c) a peroxide or peroxide-generating agent; and (d) a chelating agent.

As used herein, “procoagulant agent” may comprise one or more procoagulant compounds (e.g., chitosan, polyphosphate (“polyP”), collagen, hyaluronan, hyaluronic acid, cellulose, or kaolin). As used herein, “antimicrobial agent” may comprise one or more antimicrobial compounds. Additionally, a procoagulant agent may also have antimicrobial properties even though the procoagulant agent and antimicrobial agent are described as separate agents in portions of this disclosure.

In an embodiment, the composition of this disclosure combines a coagulating-inducing agent and an antimicrobial agent in a same mixture. Traditionally, the particular coagulating-inducing compounds described herein have not been incorporated into antimicrobial agents, both because the procoagulants serve a different purpose than the antimicrobial agents and because hydrogen peroxide, a component of some antimicrobial agents, may degrade the procoagulant properties of the procoagulant agents. In examples, the degradation may create anti-coagulant substances. However, the EDTA included in the composition described herein unexpectedly retards the conversion of hydrogen peroxide into hydroxyl radicals, thereby enabling the coagulating compounds to retain a substantial amount of their procoagulant properties, and thereby allowing for the procoagulants combination with antimicrobial components.

In embodiments, procoagulant agents described in this disclosure (e.g., chitosan, polyphosphate, collagen, hyaluronan, hyaluronic acid, and/or kaolin) can act as both procoagulants and antimicrobials. In embodiments, oxidization by hydrogen peroxide of the procoagulant agents may cause loss of both the procoagulant properties and their antimicrobial properties, and the EDTA therefore retards degradation of both properties. In an embodiment, procoagulant agents described herein can act as both procoagulants and antimicrobials when the molecular size and conformation described herein (e.g., percent acetylation of chitosan).

The addition of the procoagulant agent (e.g., chitosan, polyphosphate (“polyP”), collagen, hyaluronan, hyaluronic acid, and/or kaolin) to the antimicrobial agent presents different mechanisms of killing bacteria, fungi, spores, and viruses.

In embodiments, the composition is useful in reducing or inhibiting microbial growth, microbial infections (e.g., bacterial, fungal, viral, parasitic), microbial biofilms, inflammatory diseases, viral diseases, cardiovascular diseases, diabetes, or conditions resulting from or associated with microbial growth or infection. In embodiments, the composition is useful in treating microbial growth, microbial infections (e.g., bacterial, fungal, viral, parasitic), microbial biofilms, inflammatory diseases, viral diseases, cardiovascular diseases, diabetes, or conditions resulting from or associated with microbial growth or infection.

In another embodiment, the present disclosure provides a method of inhibiting or reducing microbial growth and reducing the loss of blood, comprising administering to a subject a therapeutically effective amount of the composition as described above.

In another embodiment, the present disclosure relates to a method of treating microbial growth and reducing the loss of blood, comprising administering to a subject a therapeutically effective amount of the composition as described above.

In another embodiment, the present disclosure relates to a method of inhibiting or reducing a microbial infection and reducing the loss of blood, comprising administering to a subject a therapeutically effective amount of the composition as described above.

In another embodiment, the present disclosure relates to a method of treating a microbial infection and reducing the loss of blood, comprising administering to a subject a therapeutically effective amount of the composition as described above.

In embodiments, the microbial growth or microbial infection is due to a microorganism comprising one or more of a bacterium, a fungus, a protozoa, or a virus.

In another embodiment, the present disclosure relates to a method of inhibiting or reducing biofilm formation and reducing the loss of blood, comprising administering to a subject a therapeutically effective amount of the composition as described above. In embodiments, the biofilm formation is the result of microbial growth, microbial infection, or contribution of host proteins, polysaccharides, lipids, and nucleic acids. Host proteins, polyphosphates, lipids, and/or nucleic acids can be host factors incorporated into a biofilm. In embodiments, the microbial growth or microbial infection is due to a microorganism comprising one or more of a bacterium, a fungus, a protozoa, or a virus. In embodiments, the composition includes multiple antimicrobial components and provides better inhibition/reduction of biofilm than a single antimicrobial agent.

In another embodiment, the present disclosure provides a method of promoting the healing of wounds.

In another embodiment, the present disclosure provides a method of promoting tissue regeneration.

In another embodiment, the present disclosure describes a method of manufacturing the composition.

In embodiments, the composition may be incorporated into a sterile packaged wound dressing and/or a hydrogel for topical application. In embodiments, the composition may be in liquid form and can be packaged in a sterile I.V.-type polymeric bag or sterile syringe, and dispensed directly onto a wound as an irrigant.

In another embodiment, the present disclosure describes a wound dressing that includes the composition, and a method of manufacturing the wound dressing.

In embodiments, the sterile antimicrobial, procoagulant composition, irrigant, and/or wound dressing could include a tourniquet or compressive device in a kit to control arterial or venous bleeding.

### BRIEF DESCRIPTION OF THE DRAWINGS

The detailed description that follows is described with reference to non-limiting and non-exhaustive embodiments shown in the accompanying figures. In the figures, the left-most digit(s) of a reference number identifies the figure in which the reference number first appears. The same reference numbers in different figures indicate similar or identical items.

FIG. 1 is a flow diagram of an exemplary method of using a composition as described herein.

FIG. 2 is a flow diagram of an exemplary method of making a composition as described herein.

### DETAILED DESCRIPTION

### Overview

The following description sets forth specific embodiments of systems and methods of use of the systems for that incorporate elements recited in the appended claims. The embodiments are described with specificity in order to meet statutory requirements. However, the description itself is not intended to limit the scope of this patent disclosure. Rather, the inventors have contemplated that the claimed disclosure might also be embodied in other ways, to include different elements or combinations of elements similar to the ones described in this document, in conjunction with other present or future technologies.

It would be desirable to develop alternative wound dressings in order to provide procoagulant properties in addition to antimicrobial properties in the same dressing.

It would be desirable for the wound dressings to have a long shelf life.

It would be desirable for the wound dressings to be easily carried in bulk or individually during times of crisis (e.g., combat, terrorist attack, natural disaster).

It would be desirable for the antimicrobial action of the wound dressings to be effective for several days or more in order to provide sufficient time, when necessary, for a patient to travel to a hospital or medical facility.

It would be desirable to combine one or more procoagulant agent(s) with one or more antimicrobial agent(s) in a composition for treating open wounds with improved activity (and with no or reduced toxicity), for optimal therapeutic use, and for developing therapeutically effective clinical regimens for these compositions. Furthermore, there is a need for formulations that are useful in a variety of related clinical indications. The procoagulant dressings could complement the therapeutic effect of tourniquets or other compression dressings for control of large vessel bleeding.

In general aspects, the disclosure pertains to fulfilling the above objectives by way of a composition for treating wounds that includes a stable mixture of one or more antimicrobial agent(s) and one or more procoagulant agent(s). Compositions, methods of manufacture, methods of treatment and/or administration, and a wound dressing incorporating the composition are encompassed by the disclosure and are depicted and described herein. Embodiments of the disclosure provide simple, safe, and effective medical treatment for wounds in resource limited environments. The present disclosure also provides other related advantages.

Unless otherwise defined, all technical terms used herein have the same meaning as commonly understood by one of ordinary skill in the art to which this disclosure belongs.

As used herein, the term “wound” refers to a type of injury in which skin is torn, cut, or punctured and/or a type of injury caused by compressive or torsional injury without penetration of the skin.

As used herein, the terms “about” or “approximate” as used in context of describing the example composition is to be construed to include a reasonable margin of error that would be acceptable and/or known in the art.

As used herein, the terms “subject,” “patient” and “individual” are used interchangeably herein, and mean a mammalian (e.g., human) subject to be treated.

As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used as described herein.

As used herein, the term “therapeutically effective amount” means an amount of a composition as described herein effective to yield the desired therapeutic response.

As used herein, the term “EDTA” refers to ethylenediaminetetraacetic acid and/or salts of EDTA (e.g., disodium EDTA, calcium disodium EDTA), when a specific form of EDTA is not explicitly recited.

The specific safe and effective amount or therapeutically effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

As used herein, the term “treatment” is defined as the application or administration of a therapeutic agent to a patient who has a wound that may or may not be actively bleeding, with the purpose to cure, heal, alleviate, relieve, alter, remedy, ameliorate, improve, or affect the wound.

Compositions and methods similar or equivalent to those described herein may be used in the practice or testing of the present disclosure. Suitable compositions and methods are described below.

The present disclosure is based on a composition representing a combination of ingredients intended to act as an antimicrobial agent and a procoagulant in medical applications. The composition of the present disclosure comprises a stable solution of various ingredients and is designed such that all ingredients in the composition are compatible with being placed on a human or animal body with no long term undesirable effects. The individual ingredients within the composition of the present disclosure are known to be safe for application onto the human or animal body in at least low levels.

The present disclosure relates to a composition representing a combination of the individual ingredients which provide coagulation and hemostasis; antimicrobial, antibacterial, antifungal, anti-inflammatory or antiviral action; antibiofilm or immunomodulatory action; or some combination of these properties.

The composition of the present disclosure comprises an antimicrobial agent (that includes an alcohol, a peroxide or peroxide-generating agent, and a chelating agent) and a procoagulant agent (in embodiments, chitosan, polyP, collagen, hyaluronan, hyaluronic acid, and/or kaolin), with the remaining balance being made up of water. The unique design feature of the composition of the present disclosure and the synergistic effects derived from the combination of the individual components provide a spectrum of effects that avoid the pitfalls of single-component treatments. For example, chitosan and polyphosphates of the molecular size and conformation (i.e., percent acetylation of chitosan) described herein, can act as both procoagulants and antimicrobials.

In embodiments, a procoagulant agent included in the composition itself has antimicrobial properties, and the composition thereby has a different antimicrobial action than with the antimicrobial agent comprising alcohol, a peroxide or peroxide-generating agent, and a chelating agent, without a procoagulant agent.

The antimicrobial agent may include water (H₂O), a non-toxic chelating agent such as EDTA, disodium EDTA, calcium disodium EDTA, magnesium EDTA, potassium EDTA, gallium EDTA, and the like, or sodium citrate; acids, salts, derivatives, or other forms of EDTA or sodium citrate; a short-chain monohydric alcohol (e.g., ethanol with a molecular formula of C₂H₅OH and an empirical formula of C₂H₆O), and a small molecule oxidizing agent such as hydrogen peroxide (H₂O₂). In an example, the antimicrobial agent may include water, EDTA, ethanol, and hydrogen peroxide.

The antimicrobial agent of the present disclosure includes an alcohol, such as a low molecular weight alcohol. The alcohol may be present in the composition at a therapeutic concentration with no or low toxicity, from about 1% to about 95% weight-to-weight (“w/w”) from about 20% to about 60% w/w, or at about 50% w/w. In embodiments, the alcohol may be present in the composition at concentrations less than 95%, 90%, 85%, 80%, 70%, 60%, or 50% w/w, and in concentrations greater than 1%, 2%, 3%, 5%, 10%, 15%, 20%, 25%, or 30% w/w. Examples of alcohols that are contemplated within the present disclosure include but are not limited to ethanol, isopropyl alcohol, n-propyl alcohol, butanol, pentanol, phenol and phenol derivatives, furanol and furanol derivatives, diols, triols, polyols, including chain, ring and aromatics, and the like. In an embodiment, the antimicrobial agent comprises ethanol, at about 50% of the composition weight to weight.

The antimicrobial agent of the present disclosure also includes a peroxide or peroxide generating agent. In embodiments, it may be present in the composition at a therapeutic concentration with no or low toxicity, from about 0.025% to about 20% w/w, from about 0.05% to about 10% w/w, or at about 1.5% w/w. In embodiments, the peroxide or peroxide generating agent may be present in the composition at concentrations of less than 20%, 15%, 10%, 5%, 2%, or 1% w/w, and in concentrations greater than 0.025%, 0.05%, 0.10%, or 0.20% w/w. Examples of peroxide or peroxide-generating agents that are contemplated within the present disclosure include but are not limited to hydrogen peroxide (H₂O₂), carbamide peroxide (i.e., urea peroxide), peroxy acids such as peroxyacetic acid, peroxybenzoic acid, acetic anhydride, and the like. In some cases, free-hydroxyl or free-radical generating substances could be present or a substitute for hydrogen peroxide. Examples of free-radical generating substances that are contemplated within the present disclosure include but are not limited to acetone peroxide, t-butyl peroxide, di-t-butyl diazine ((t-Bu)₂N₂), and the like. Other free-radical generating materials include those which generate free radicals on exposure to, for example, UV light. In an embodiment, the antimicrobial agent comprises H₂O₂, at about 1.5% of the composition w/w. The slow release of oxygen from the peroxide-generating agent when the composition is applied potentially increases tissue oxygenation, epithelialization, revascularization, and potentially prevents colonization or infection by anaerobic bacteria.

In embodiments, the peroxide or peroxide-generating agent in the composition comprises hydrogen peroxide (H₂O₂). In embodiments, the H₂O₂ may be present at a therapeutic concentration with no or low toxicity, from about 0.05% to about 40% w/w. In other embodiments, the H₂O₂ is present at a concentration of from about 0.05% to about 10% w/w. In alternative embodiments, the H₂O₂ is present at a concentration of about 0.25% w/w. In embodiments, the H₂O₂ may be present in the composition at concentrations of less than 10%, 9%, 8%, 6%, 4%, 3%, 2%, 1%, 0.50%, 0.25%, or 0.20% w/w, and in concentrations greater than 0.025%, 0.05%, 0.10%, 0.20%, or 0.25% w/w.

The antimicrobial agent of the present disclosure further includes one or more compounds that are chelating agents (chelators). They are present in the composition of the present disclosure in the form which results when the pH of the solution is adjusted to the desired level for a particular application. Examples of chelating agents that are contemplated within the present disclosure include but are not limited to ethylenediamine tetraacetic acid (EDTA), citrate, and their salts, other substituted compounds, such as salicylic acid or salicylate esters, and the like. In an embodiment, the antimicrobial agent comprises EDTA, at a therapeutic concentration with no or low toxicity, from about 5 mg/mL to about 50 mg/mL, or about 10 mg/mL.

While not wishing to be limited by any particular theory, chelators are primarily known to act to form strong bonds to a wide variety of inorganic or organic ions, thereby rendering them relatively unavailable for use in metabolic processes of various kinds. Specifically, the ions are thereby prevented from binding by or use by certain proteins and/or enzyme systems to support or cause specific processes in metabolic action. Exemplary proteins known to bind various ions are metal metalloproteinases (MMPs), which bind divalent cations such as zinc (Zn+2). The chelating effect of agents such as EDTA may inhibit the activity of MMPs by depriving the MMP of the Zn+2 ion (which is required for its function). Thus, the chelating agents (such as EDTA) in the composition of the present disclosure may assist in controlling, inhibiting, or avoiding tissue destruction caused by MMPs. Metallocarbamases also require divalent cations, e.g., zinc, and may be another target for EDTA or similar chelators. Other metalloproteases also are contemplated. EDTA modulates cytokine and matrix metalloproteinase over reactivity, especially in the acute phase traumatic or burn wound.

Zinc finger proteins are generally found as DNA binding protective proteins. They contain one or more short loop(s) with a conserved His-Cys motif binding generally one zinc ion per loop. They provide the DNA protective function by enwrapping the DNA molecule with a protein “glove” which in turn is held in place by one or more intercalated zinc fingers spaced along the DNA helix. Generally, zinc finger formation and stability may be disrupted by chelators such as EDTA, by binding and controlling the amount of free Zn+2, thus providing a means by which the composition has the potential to modify the degree of DNA protection from or exposure to other agents.

In addition, it is known that divalent ions, specifically Mg+2 and Ca+2 ions, must be present for the formation and/or maintenance of the exopolysaccharide matrix that forms the bulk of biofilms. It is further known that EDTA imbues a solution with the power to disrupt or completely disintegrate an existing biofilm, and to retard or prevent their formation at least in the case of some medically significant biofilm forming organisms. It is therefore reasonable to suggest that inclusion of EDTA or other chelators in the composition of the present disclosure, especially along with the other active ingredients, will be synergistically useful in biofilm prevention and eradication.

It is contemplated that, in an embodiment, calcium is incorporated into a formulation of chitosan or onto chitosan's surface via charge control. In an example, Ca-EDTA complex might be attached to chitosan or modified chitosan to deliver the chelating agent to areas where calcium or more intense clotting response is needed.

It is contemplated that, in an embodiment, the cationic nature of chitosan interacts with the steroidal components to influence the degree of germination in intestinal areas.

In embodiments, EDTA can be used as the chelating agent, or EDTA's salts (e.g., for example, the disodium salt, the calcium disodium salt, dipotassium salt, tetrasodium salt). In all these cases and others, the actual ionic composition of the EDTA in the composition of the present disclosure will adjust, with the EDTA acting as a buffer, as the pH is adjusted. Furthermore, the EDTA provide a protective function in the composition of the present disclosure, as in other known cases, by protecting the peroxide from divalent-catalyzed decomposition.

Although EDTA has a relatively low solubility in solutions of, for example, ethanol, peroxide or peroxide-generating agents such as hydrogen peroxide (H₂O₂) can act as a powerful co-solvent. In an example, in solutions of 50% w/w ethanol or higher, and at near-physiological pH but in the absence of H₂O₂, disodium EDTA's solubility is limited to less than 10 mg/mL. In another example, when H₂O₂ is present, even at levels as low as 1-2%, stable solutions of disodium EDTA at 10 mg/mL and at least 50-60% w/w ethanol are readily prepared and are stable (i.e., no precipitation or other changes are seen) at temperatures as low as 0° C. Furthermore, even in solutions of 50% w/w ethanol, disodium EDTA in the presence of 6% H₂O₂, remained soluble at concentrations as high as 40 mg/mL, both at 27° C. and at 0° C. This unexpected effect is not due to the presence of additional water introduced with the H₂O₂, as evidenced by the fact that the effect occurs even when the total amount of water in the solution is held constant.

Thus, the unique combination of the individual ingredients in the composition of the present disclosure provides relatively low concentrations of ethanol, low concentrations of EDTA, and hydrogen peroxide levels that are unavailable by other means. The composition of the present has unexpected stability yet can be functionally powerful and versatile as an antimicrobial agent. Stability studies have shown shelf life lives (at room temperature) of 4% H₂O₂ solutions with relatively low ethanol concentrations and low EDTA concentrations in excess of 14 months while retaining essentially all of the peroxide and alcohol effectiveness. Hydrogen peroxide solutions may have active lifetimes of multiple months or multiple years. The unexpected stability of EDTA against precipitation provided by the H₂O₂, and the stability of the hydrogen peroxide against decomposition, provided by EDTA is an example of synergy not normally seen in inanimate systems and resembles a symbiosis relationship. Additional chelators may be used to adapt the composition of the present disclosure to specific applications. Examples of these may include but are not limited to citrate, pyridine derivatives, various diamines or substituted diamines, and the like.

In addition to the stability, the relatively low concentrations of alcohol (such as ethanol) in the composition of the present disclosure is able to deliver its killing power essentially unmitigated because of the unique properties conferred by the peroxide or peroxide-generating agent (such as H₂O₂) and the chelating agent (such as EDTA) in the combination. These concentrations are therapeutic and have low to no toxicity and reduce alcohol absorption into blood stream and avoid damage to open wounds. For example, some evidence exists that suggest penetration into a biofilm by H₂O₂ might be reduced because of the presence of peroxide-reactive agents, e.g., bacterial catalase or superoxide dismutase, near the outer (distal) surface of the biofilm. However, with inactivation of the peroxidases by the chelating agent (such as EDTA), the H₂O₂ is able to penetrate deeply into the biofilm and eradicate the microorganism. The presence of ethanol in the composition of the present disclosure may help increase the H₂O₂ penetration by disrupting lipid, lipopolysaccharide, and protein barriers in the biofilm. In some medical conditions, e.g., Candida albicans, the presence of ethanol may significantly reduce the thickness of the biofilm via the presence and action of the alcohol dehydrogenase (ADH) enzyme. Further, ethanol is present in large concentration relative to both the enzyme and its cofactor, NADH. The consumption of ethanol will be severely limited, particularly because one of the mechanistic steps in the alcohol dehydrogenase utilizes free Zn+2 ion. Thus, in the combined action of the composition, EDTA—tying up zinc and simultaneously attacking the biofilm structure itself with limited NADH cofactor for ADH—only a small fraction of the total ethanol will be consumed, leaving the bulk remainder to act to penetrate the biofilm rapidly, delivering its killing power. Even lower concentrations of ethanol allow penetration through lipid and protein wound debris, in addition to bactericidal, viricidal, and fungicidal activity.

In addition to the antimicrobial agent described above, the composition includes a procoagulant agent.

Procoagulants are a type of topical hemostatic agent. Procoagulants function by activating the clotting cascade or delivering high concentrations of procoagulant factors to a bleeding wound. In an example, when applied, the procoagulant agent described herein promotes vascular hemostasis.

In an example, the procoagulant agent comprises chitosan at therapeutic levels. At certain molecular weights (discussed below), chitosan has procoagulant properties and has been used as a coagulant in would dressings to prevent traumatic or post-operative bleeding. Chitosan is a natural polycationic linear polysaccharide derived from partial deacetylation of chitin and is chemically composed of 2-Amino-2-deoxy-β-D-glucopyranosyl-(1->4)-2-amino-2-deoxy-β-D-glucopyranosyl-(1->4)-2-amino-2-deoxy 43-D-glucopyranosyl-(1->4)-2-amino-2-deoxy-β-D-glucopyranosyl-(1->4)-2-amino-2-deoxy 43-D-glucopyranosyl-(1->4)-2-amino-2-deoxy-β-D-glucopyranosyl-(1->4)-2-deoxy-2-[(methoxycarbonyl)amino]-β-D-glucopyranosyl-(1->4)-2-amino-2-deoxy-β-D-glucopyranosyl-(1->4)-2-amino-2-deoxy 43-D-glucopyranose. The chitosan creates blood clotting primarily because positively-charged chitosan attracts negatively-charged red blood cells and platelets, which shortens the clot formation in addition to hemostasis time. Chitosan's cationic nature is induced by positively charged amino groups; these groups induce fibrinogen adsorption, activation of platelets, release of coagulation factors, and then trigger extrinsic coagulation pathway.

Hydrogen peroxide, a component of the composition described herein, oxidizes large molecule chitosan (generally not water-soluble) into lower molecular weight chitosan (generally highly water-soluble), which has non-coagulant or anti-coagulant properties If hydrogen peroxide degrades the chitosan to oligosaccharides, it may diminish the procoagulant activity of the chitosan. Thus, while chitosan could be desirable to include in an antimicrobial agent because it is procoagulant, chitosan has not previously been included because of its oxidation in the presence of hydrogen peroxide into an anti-coagulant resembling heparin.

It is believed that this problem is ameliorated by the presence of the EDTA in the composition. The EDTA counteracts the oxidation of the chitosan by retarding conversion of hydrogen peroxide into hydroxyl radicals. In embodiments, hydroxyl radicals may oxidize the chitosan into an anticoagulant. EDTA mitigates the conversion of hydrogen peroxide to hydroxyl radicals, which could convert chitosan to chitooligosaccharide.

The composition described herein may stop bleeding in fresh wounds and prevent or eradicate a broad spectrum of antimicrobial resistance (AMR) pathogens.

In addition to its procoagulant properties, chitosan inhibits or kills bacteria, fungi, and some viruses. Chitosan gauze field dressings are used by the United States military for this purpose. Chitosan is also known for its tissue healing effect on wounds. Chitosan's inclusion in the composition is helpful in the subacute/intermediate and chronic/healing stages for promoting wound closure with epithelial cells.

In embodiments, chitosan also has other functions in the composition, with or without the addition of other reagents that react with chitosan. Additional contemplated functions and/or activity include but are not limited to bacterial spore reactions in the presence of bile acids, bone tissue scaffolding and bone healing (e.g., in combination with polyvinyl alcohol), treatment of chronic osteomyelitis after infection of a wound, and topical treatment of cancer and diabetic ulcers.

Chitosan may be made by treating the chitin shells of shrimp and other crustaceans with an alkaline substance. However, chitosan made by other methods (such as synthetically) and from other sources could be used. Chitosan's molecular size can vary. In embodiments, the molecular size of chitosan may be dependent on the source of the chitin from which it is derived (e.g., crab shells, shrimp, fungi, etc.). On average, the molecular weight of commercially produced chitosan may be between 3,800 and 20,000 Daltons (Da). In embodiments, the molecular weight of chitosan described as being used as a procoagulant herein may be from about 310 kilodaltons (kDa) to 375 kDa, or from about 700 kDa to about 1000 kDa, or from about 50 kDa to about 500 kDa. In embodiments, the molecular weight of chitosan described as being used as a procoagulant herein is less than 1000 kDa, 750 kDa, or 500 kDa and is greater than 50 kDa, 310 kDa, 400 kDa, or 500 kDa. The physical properties of chitosan in aqueous solution depend on the degree of deacetylation and the acetyl group distribution in the polymer chains.

It is contemplated that the composition could replace conventional antimicrobials, which is advantageous because, for example, widespread use of chlorhexidine gluconate (CHG) for routine washing and wiping of pre-operative sites, has led to the increased incidence of resistant Staphylococcus aureus, both to methicillin (MRSA) and CHG, in some hospital environments.

In examples, the composition may be used as an antimicrobial for extremely drug resistant (XDR) pathogens and/or multidrug resistant (MDR) pathogens at least because (1) the procoagulant agent (e.g., chitosan) may be effective against gram-positive bacteria, and/or (2) the composition has at least four antimicrobial agents (e.g., hydrogen peroxide, ethanol, procoagulant (e.g., chitosan), EDTA) that have four different chemical mechanisms for antimicrobial action. In an example, the composition may be used to treat chronic osteomyelitis. Chronic osteomyelitis with MDR bacterial or fungal pathogens can lead to extremity amputations in combat, conflict, disaster, and trauma victims. Topical or injection of the composition into an infected bone may stop infection and promote healing.

In embodiments, chitosan may be chemically modified or incorporated into composites or forms (including, but not limited to, hydrogels, fibers, gels, liquids, or nanoparticles), that yield additional functional properties for different applications. Chitosan has been blended with other macromolecular polymers or biomaterials such as cellulose acetate, lysozyme, bentonite, and silk fibroin to enhance its functionalities as a wound-care dressing material. The hydroxyl and amine groups of chitosan may be functionalized under mild reaction conditions to prepare modified chitosan derivatives with altered, desirable properties.

In an example, polyvinyl alcohol and chitosan may form hydrogels. In examples, polyvinyl alcohol (or salts of polyvinyl alcohol) and polyglactic acid may both bond to chitosan. In another example, quaternary ammonium chitosan/polyvinyl alcohol/polyethylene oxide hydrogels prepared with the use of gamma radiation exhibit desirable swelling ability, water evaporation rate as well as mechanical characteristics and high antibacterial potency against E. coli and S. aureus, and therefore, it is contemplated that such hydrogels can promote healing in wound dressings.

In an example, chitosan fibers can be incorporated into a gauze dressing with antimicrobial and procoagulant properties.

In embodiments, chitosan can form composites with (1) natural polymers (e.g., collagen, gelatin, alginate, silk fibroin), and/or (2) synthetic polymers (e.g., poly-caprolactone (PCL), poly-lactic acid (PLA)), and/or (3) bioceramics (e.g., calcium phosphate (CPC), calcium polyphosphate (CPP), hydroxyapatite (HA). Li. These composites can be used for tissue regeneration and/or articular cartilage defect repair. Li. In an example, a composite of chitosan and calcium polyphosphate is used in the composite described herein. The CPP is biodegradable and can eventually be replaced by the repaired tissue. Id. CPP degrades into calcium and phosphate, which are essential to organisms and do not induce inflammation. Id. It is contemplated that a chitosan/CPP scaffold would integrate with adjacent tissue. Id.

In an embodiment, chitosan can be blended with phosphate groups to form phosphorylated chitosan conjugates that can act as procoagulant agents. As described in Wang, chitosan can be tethered to phosphate groups to mimic polyP. Wang, Y., et al. Hemostatic Ability of Chitosan-Phosphate Inspired by Coagulation Mechanisms of Platelet Polyphosphates. Macromolecular Bioscience. 2018. doi: 10.1002/mabi.201700378.

In embodiments, the composition includes chitosan in the form of lower molecular weight-water soluble chitosan, or chitosan conjugated polylactic acid. The structure of polyP consists of linear polymers of orthophosphate linked by high-energy phosphoanhydride bonds. Generally, the polymer length of polyP may range from several phosphates to several thousand phosphate units (from about 3 polyphosphate units long to about 6,000 phosphate units long, or from about 5 polyphosphate units long to about 4,000 phosphate units long, or from about 20 polyphosphate units long to about 3,000 phosphate units long).

In an embodiment, the procoagulant agent used in the composition of the present disclosure may comprise microbial long-chain polyphosphate (“long-chain polyP”) at therapeutic levels.

The molecular weight of polyp generally increases as length increases. The roles of polyP in blood clotting vary depending on polymer length.

Long-chain polyP ranges from less than a hundred phosphates to several thousand phosphate units long. Microbial long-chain polyP (ranging from less than a hundred phosphates to several thousand phosphate units long) acts at four points in the clotting cascade (initiates the contact pathway of blood clotting; accelerates factor V activation and abrogates TFPI function; enhances fibrin polymerization; accelerates factor XI back-activation by thrombin.)

In another embodiment, platelet-size polyP (about 60 to 100 phosphate units long) may be used as the procoagulant agent. Although platelet-derived polyP (and synthetic polyP of the same size, i.e., 60-100 mers), may trigger clotting via the contact pathway, it is less potent than long-chain polyP. Platelet-size polyP acts most potently at three points in the clotting cascade: abrogates TFPI function (and overlaps the minimal size necessary to accelerate factor V activation); overlaps the minimal size necessary to enhance fibrin polymerization; accelerates factor XI back-activation by thrombin.

PolyP may be chemically synthesized in large quantities by heating sodium orthophosphate to a few hundred degrees Celsius and then rapidly cooling. Water-soluble, high molecular weight polyP may be isolated from very high molecular weight “insoluble” polyP by differential solubilization using LiCl solutions. Other size ranges of polyP may be prepared via partial alkaline or acidic hydrolysis of long-chain polyP, enzymatic degradation with endopolyphosphatases or differential acetone precipitation under various salt conditions.

In an embodiment, the polyP used in the composition described in this disclosure is long-chain polyphosphate (“long-chain polyP”), which is a natural pathophysiologic activator of the contact pathway of blood clotting.

In a similar process as that described above for chitosan, long-chain polyP may be oxidized by hydrogen peroxide which may inactivate its clotting property. The EDTA in the composition described herein may prevent the production of hydroxyl radicals and preserve the clotting nature of long-chain polyP, allowing it to be an effective procoagulant in the composition described herein that includes an antimicrobial agent. PolyP itself may also have antimicrobial properties.

In an embodiment, the procoagulant agent comprises kaolin. Kaolin acts as a procoagulant by surface activation of platelets and the release of polyP. Kaolin can be incorporated into a sponge, gel, and/or fabric type wound dressing product.

In an embodiment, the procoagulant agent comprises collagen at therapeutic levels. Collagen can be incorporated into a sponge, gel, and/or fabric type wound dressing product. Additionally or alternatively, collagen-calcium polyphosphate can help in the healing of wounds. In an embodiment, calcium EDTA, rather than disodium EDTA, is used in a composition that includes collagen. In an embodiment, host-guest composite particles can be prepared from amorphous collagen (host) and polyphosphate (guest), that can increase the wound-healing rate.

In an embodiment, the procoagulant agent comprises hyaluronan or hyaluronic acid. The procoagulant properties of hyaluronic and hyaluronic acid can vary with the molecular weight of hyaluronan. In embodiments, high molecular weight hyaluronic acid or hyaluronan may be a component of the composition. High molecular weight hyaluronic acid or hyaluronan may have a molecular weight of greater than 500 kDa or greater than 800 kDa Hyaluronan and hyaluronic acid promote wound healing. Hyaluronic acid can increase levels of certain growth factors (e.g., TGF-β1, PDGF) released by platelet-rich plasma (PRP) and can result in enhanced healing effect of certain tissues. Iio, K., et al. Hyaluronic Acid Induces the Release of Growth Factors from Platelet-Rich Plasma. Asia-Pacific Journal of Sports Medicine, Arthroscopy, Rehabilitation and Technology. 2016. doi: 10.1016/j.asmart.2016.01.001.

In embodiments, a single procoagulant agent can be used in the composition. In other embodiments, combinations of two or more of the procoagulant agents described herein may be used in the composition. It is contemplated that a combination of the two or more procoagulants could be used when the mechanisms are either compatible or synergistic. The two or more procoagulant agents can be individual components of the composition, or can form a composite before or after being added to the composition.

In an example, both chitosan and polyP may be used in the composition. In an example, both soluble and insoluble chitosan-polyP have procoagulant properties. In an embodiment, it is contemplated that chitosan and polyP may be used in the composition by encapsulating one of the procoagulants in gel beads or in liposomal particles.

In embodiments, chitosan, collagen, and hyaluronic acid can form biocompatible, porous structures that can be used as matrices for calcium phosphate in situ precipitation. The composite materials prepared by calcium phosphate precipitation in a chitosan/collagen/hyaluronic acid sponge cross-linked by dialdehyde starch can be used for tissue engineering and regeneration. Id. It is contemplated that these composite materials can be used in the composition described herein, e.g., as a dressing, gel, irrigant, scaffold, for use as filler for bone and cartilage, regenerating joint or skin, treating septic arthritis and osteomyelitis, and dental bone reconstruction, e.g., osteomyelitis.

In embodiments, certain RNA homopolymers (e.g., polyG and polyI) can also be used as a procoagulant agent at concentrations ranging from about 10 to about 20 ug/mL, from about 8 to about 12 ug/mL, or from about 5 to about 15 ug/mL.

Additionally, in embodiments, other combinations of the procoagulant agents described herein may be used in the composition with compatible or synergistic effects. Smith, S., et al. Ability of Polyphosphate and Nucleic Acids to Trigger Blood Clotting: Some Observations and Caveats. Frontiers in Medicine. 2018. doi: 10.3389/fmed.2018.00107.

In embodiments, the procoagulant may be present in the composition at a non-toxic concentration of from about 1% to about 95% w/w, from about 20% to about 60% w/w, or at about 50% w/w. In embodiments, the alcohol may be present in the composition at concentrations less than 95%, 90%, 85%, 80%, 70%, 60%, or 50% w/w, and in concentrations greater than 1%, 2%, 3%, 5%, 10%, 15%, 20%, 25%, or 30% w/w. The concentration of the procoagulant may be variable, dependent on the particular application for which the composition is being used.

Additional ingredients—beyond the antimicrobial agent and the procoagulant agent—may be included in the composition of the present disclosure.

It may be necessary to adjust the pH of the composition for particular applications. For example, in applications where the composition might be acidic on production, a base, typically but not exclusively sodium hydroxide solution, may be added to adjust the pH to the desired pH or to physiological pH. Alternatively, if the composition is basic when produced, an acidic agent, typically but not exclusively either hydrochloric acid, hypochlorous acid, citric acid, ascorbic acid, phosphoric acid, or acetic acid, may be added to return the pH to the desired pH or to physiological pH. In embodiments, it might be desirable for the composition to be at some non-neutral or non-physiological pH, in which case additional adjustments would be made. Acetic acid, acetate salts, citric acid, citrate salts, and phosphoric acid themselves have antimicrobial activity and so serve both functions of pH adjustment and antimicrobial activity in an embodiment of the composition. In addition, citrate has antibacterial activity. In embodiments, it is contemplated that an acidic agent (e.g., hypochlorous acid) in the composition described herein can keep the hydrogen peroxide more active.

Thus, in embodiments, the composition described herein may comprise four antimicrobial components (i.e., EDTA, low molecular weight alcohol, peroxide or a peroxide-generating agent, and at least one of acetic acid, acetate salts, citric acid, citrate salts, or phosphoric acid). In embodiments, a procoagulant agent is a fifth antimicrobial component of the composition. In embodiments in which more than one procoagulant agent described herein is used in the composition, the additional procoagulant agents can have antimicrobial properties.

A viscosity-increasing agent (such as a thickener or gelling agent) might also be desirable to include in the composition. Examples of viscosity-increasing agents include but are not limited to carboxymethyl cellulose (CMC), hydroxypropyl methylcellulose (HPMC), methyl cellulose, methyl hydroxyethyl cellulose (MHEC), hydroxyethyl cellulose, sodium hydroxyalkyl celluloses, and admixtures thereof. Other viscosity-increasing agents are contemplated, among them, but not limited to, silicone-based products such as dimethicone and silicone gels. In an embodiment, the comprises hydroxypropyl methylcellulose (HPMC) at a concentration of from about 0.25% to about 2% w/w.

The composition may also provide a means by which other potentially harmful components in a combat wound, trauma, burn or other healing areas are inactivated, and tissue injury and systemic toxicity are reduced. These components comprise one or more of host MMPs, TNF-alpha, bacterial beta-lactamases, (including multi-resistant extended spectrum beta-lactamases (ESBL)), carbapenemases, or metallocarbamases. The components are generally relatively complex protein-based compounds. However, the composition of the present disclosure provides a spectrum of biochemical reactions, at least one or more of which will be effective at disrupting and/or attenuating the harmful processes. Healing rates and/or the general well-being of the subject will be improved by a reduction in the adverse effects seen from host over-expression of cytokines, chemokines, and other inflammatory molecules. Preservation of therapeutic activity of systemic antimicrobials, by preventing microbial enzymatic inactivation and lowering of bacterial and fungal toxins, will also augment host survival.

The balance of the composition will be made up of water.

In embodiments, one or more of the components of the antimicrobial agent and one or more of the components of the procoagulant agent may be covalently linked together or separate from each other. In an example, chitosan, collagen and hyaluronic acid may be covalently linked.

The composition (such as strength of ingredients) may be tailored to the specific needs of an individual. For instance, the composition may be dependent upon such factors as nature of the injury, depth of the wound, location of the wound, duration of time expired post injury, superinfection, etc.

The composition of the present disclosure may be in solution form in certain applications. In other applications, the composition of the present disclosure may be in other forms, such as gel, cream, ointment, drops, spray, slurry, and the like. The composition of the disclosure is not limited to a particular means of application or to any particular state (liquid, gel, slurry, etc.). In examples, the composition may be contained in a wound dressing, or may be in the form of a hydrogel or liquid. The means of application and/or state of the composition can be dependent on type of wound, location of wound, extent of wound, and situation. In an example, a flexible plastic bag can be filled with the composition, in solution, to flush out a wound that is hyperacute or acute. Dressings that are for extended use when desirable (e.g., evacuation from battlefield, in limited resource disaster areas). Another type of dressing can be used in acute or subacute care in wound clinics or hospitals. Another type of dressing can be used for chronic/regenerative stage wounds (e.g., for prescribing in outpatient clinics, chronic care facilities, rehabilitation facilities, home care). Depending on the wound status or situation, dressings can be impermeable, semipermeable to vapor and/or liquid, or permeable to liquid and/or vapor. In an example, when a patient is being transported long distances, a dressing that maintains a hydrated wound environment can be used.

In embodiments, chitosan may link with other molecules to form chitosan hydrogels, which may be incorporated into the composition to prevent or treat diabetic skin ulcer infections, i.e., on the legs. Microvascular disease and peripheral neuropathy are frequent components in diabetic ulcers.

In an example, the composition the present disclosure may be applied in conjunction with medical dressings. In an example, the dressing material may be a non-toxic material that will release the composition into the medical areas as desired. Appropriate dressing materials will depend upon the nature of the injury and the overall condition of the patient.

In an embodiment, the composition may be applied to a wound dressing and the dressing may be then placed in sterile packaging. The packages that result may be distributed to civilians, the military, medical professionals, etc.

In an embodiment, a package containing the composition may include an affixed barcode. A barcode may represent data about the manufacture and contents of the package. At the time of application of the wound dressing, the barcode may be scanned by a smartphone, handheld scanner, etc., to capture data regarding the administration of the wound dressing to be associated with the barcode. Reports may be run from the data collected and associated with the barcode may be run for any number of purposes, such as tracking batches of the composition.

The specific concentration, components, and delivery method of a wound dressing that includes the composition herein may be dependent upon wound staging, degree of infection, size of vessel, etc. Wounds may be classified in different ways depending on the characteristic of the wound being assessed, such as (1) acuteness (e.g., Hyperacute (immediately post injury), Acute, Subacute, Healing, and Chronic); (2) degree of infection/microbial load (infected/presumed infected; intermediate biofilm and microbial infection; low microbial colonization, etc.); (3) healing potential (clean wound, post-operative, low risk contamination, etc.); (4) type of blood vessel; (5) volume of loss; (6) area of exposure. In an example, it is contemplated that a dressing including the composition as described herein, together with a bandage wrap or blood pressure cuff type compression, could be effective for hyperacute or acute battlefield wounds. If there is large vessel bleeding, tourniquets (e.g., windless, elastic, basic tubing), direct compression, and/or surgery would be most appropriate.

In an example, if the wound is a combat blast wound with small vessel bleeding and environmental particulate and microbial contamination, the wound may be irrigated with a sterile solution of the composition with a lower-range concentration of EDTA to avoid anticoagulant effect and lower-range concentration of ethanol to decrease pain of irrigation, and lower-range hydrogen peroxide to avoid excessive absorption, but still add an hemostatic effect by platelet clumping and blood clotting.

Later stages of wound healing usually would not require the procoagulant activity of the composition unless there is recent surgical debridement; however, one or both of the procoagulant agents described herein could be retained for beneficial antimicrobial or wound healing attributes. Additionally, the composition of the present disclosure can promote tissue regeneration by way of the components comprising chitosan, collagen, polyphosphate, hyaluronan and/or hyaluronic acid, and/or others like mesenchymal stem cells or interstitial substrate. The composition may contribute to tissue regeneration by promoting wound healing and/or preserving tissue. For example, chitosan and collagen polymer scaffolds, as well as calcium phosphate ceramic and other bioceramics, can be used to support regenerating tissue. Li. The bioactivities of chitosan help to reduce inflammation when chitosan scaffolds are used (e.g., for articular cartilage defect repairs). Id. It is contemplated that chitosan is biocompatible towards human skin. Id.

Beyond its antimicrobial properties, the hydrogen peroxide in the composition may act as a hemostatic agent and promote the healing of wounds. Hydrogen peroxide may assist in balancing (1) the destructive processes of wound healing that remove damaged tissue and (2) the repair processes of wound healing which lead to new tissue formation. Hydrogen peroxide may promote oxidative stress and resolve inflammation, which makes it a bidirectional regulator. Hydrogen peroxide may facilitate hemostasis at least by regulating the contractility and barrier function of endothelial cells, activating latent cell surface tissue factor and platelet aggregation, and stimulating platelet derived growth factor activation. The activity of hydrogen peroxide may be prolonged in the composition because the EDTA in the composition may decrease the activity of catalase by chelating its divalent cation. In embodiments, the hydrogen peroxide in the composition functions as a signaling agent and has immunomodulatory effects.

The composition of the present disclosure may be administered as a single treatment or as multiple treatments over a period of time depending on the overall condition of the patient and medical attention needed.

The composition of the present disclosure may be administered by many means including but not limited to topical application.

The composition of the present disclosure may be administered to many areas including but not limited to a wound site (including skin around wound areas), a surgical site, an injection site, a thermal burn site, a chemical burn site (e.g., from nitrogen mustards), an electrical burn site, a radiation burn site, a skin lesion (abrasions), oral sites (such as leukoplakias, carcinomas-in-situ, oral carcinomas, aphthous ulcers (i.e., open lesions), bony sites (with osteomyelitis, for example, caused by Staphylococcus aureus, Pseudomonas aeruginosa, Acinetobacter baumannii), anal sites, vaginal sites, cervical sites, vulvar sites, penile sites, ulcerated skin sites (e.g., diabetic foot ulcers, decubiti (“bed sore”) sites), acne sites (e.g., facial, trunkal, and others), actinic keratosis sites, inflamed sites, irritated sites, gastric sites, gastrointestinal sites (upper and lower), esophageal sites, esophagogastrointestinal sites, intestinal sites, cardiac sites, vascular sites, nasal sites, nasopharyngeal sites, and aural sites and may be included in a catheter locking solution.

The composition of the present disclosure may be provided to inhibit or reduce microbial growth, inhibit or reduce microbial infections, inhibit or reduce, and/or inhibit or reduce microbial biofilms. The microbial growth, microbial infections, and microbial biofilms may be due to different microorganisms including but not limited to a bacterium, a fungus, a protozoa and a virus.

It is contemplated that the composition of the present disclosure may be provided to sites such as, without limitation, wounds, burns, skin lesions and vesicles, oral, cervical, vaginal, vulvar, penile, anal sites, esophageal sites, and the like.

The composition may also be provided to modulate cytokines that are overexpressed in radiotherapy.

The composition may also be provided to treat a viral condition or disease. The role of viruses (and viral infections) in the pathogenesis of cancers is another important medical research area. Human papilloma virus (HPV) is a member of the papillomavirus family of viruses that is capable of infecting humans. HPV infections occur in the stratified epithelium of the skin or mucous membranes (such as in the cervix, vulva, vagina, penis, anus and oropharynx). Persistent infection with “high-risk” HPV types may progress to precancerous lesions and invasive cancer. A growing number of studies have shown a link between HPV infection and certain types of cancers (such as penile and anal cancers). Further studies have also shown a link between a wide range of HPV types and squamous cell carcinoma of the skin. It is contemplated that effective treatment of the leukoplakia condition (a form of squamous cell carcinoma) using the composition of the present disclosure may be mediated, in part, by its effect on any possible underlying viral infection or activity. The effect may also be mediated by any possible underlying inflammatory activity.

The composition of the present disclosure may further provide a means of significantly reducing the severity and shortening the course of “cold sores” likely resulting from an outbreak of Herpes simplex. Accordingly, the composition of the present disclosure may also be provided to inhibit or reduce a viral condition or disease.

Another contemplated application of the composition of the present disclosure may be for the inhibition and/or treatment of canker sores (aphthous ulcers), a type of oral ulcer, which presents as a painful open sore inside the mouth or upper throat and is characterized by a break in the mucous membrane. Once thought to be a herpes virus infection, the entire class of canker sores is now thought to be an aggregate of a variety of disease processes, each with the ability, in its own way, to produce rapid but self-limiting destruction of mucous membranes, predominantly through immunologic and ischemic mechanisms. In some individuals the ulcers are a secondary or hypersensitivity response to antigenic stimulus, especially foods), while in others they are a primary autoimmune disorder. It is contemplated that the composition of the present disclosure may be useful for the inhibition and/or treatment of canker sores (aphthous ulcers). The effect may be mediated by modulation of any underlying viral infection or activity or inflammatory activity.

It is contemplated that the composition of the present disclosure may be used as adjunctive therapy in combination with existing therapies. The term “adjunctive” is used interchangeably with “in combination” or “combinatorial” and are used where two or more therapeutic or prophylactic regimens affect the treatment or prevention of the same condition.

The composition of the present disclosure may provide a means for altering the chemical environment of target areas of human and animal patients, especially in wounds, burns, surgical sites, and catheter insertion sites, to prevent cell damage and/or toxicity by the presence of a range of materials that are commonly found at such sites. Such materials often have a balance of beneficial and deleterious effects, depending on their concentrations and other factors. They are usually present in very low levels, generally in the micromolar or even at the nanomolar levels (10⁻⁹) levels, and in some cases picomolar (10⁻¹²) levels. Accordingly, this application provides a series of reactive possibilities at such levels because the combination of components in the composition are present at significantly higher levels (i.e., millimolar or molar), which has the effect of driving reactions, which would otherwise seem to be unfavorable, further towards completion.

In an embodiment, ethanol and hydrogen peroxide, when included in the composition of the present disclosure, can inactivate bacterial endotoxins (e.g., LPS), exotoxins, and enterotoxins. In an example, the procoagulant agent (e.g., chitosan, polyP) can be effective against some gram-positive and/or gram-negative bacteria. Inactivation of bacterial endotoxins (e.g., LPS), exotoxins, and enterotoxins can be important in preventing a cytokine cascade, septic shock, multiorgan system failure, etc.

Different types of wound infections can be promoted by different bacterial toxins. Examples of anaerobic bacteria in wounds that could be inactivated by the composition of the present disclosure include but are not limited to Clostridium tetani (tetanus), Clostridium perfringens (gas gangrene), and Bacteroides fragilis (synergistic necrotizing fasciitis), etc. Examples of aerobic bacteria in wounds that could be inactivated by the composition of the present disclosure include but are not limited to gram positive, Staphylococcus aureus (Toxic Shock Syndrome from TSST-1 Streptococcus hemolyticus), Group A, Beta hemolytic (ascending cellulitis), lymphangitis Streptococcus faecium (vancomycin resistant—cellulitis or abscess). Examples of gram negative bacteria in wounds that could be inactivated by the composition of the present disclosure include but are not limited to Pseudomonas aeruginosa, Acinetobacter baumannii, which both can be multidrug resistant (MDR) pathogens and produce cellulitis, abscesses, septicemia, and septic shock. In an embodiment, chitosan (cationic) can be used as an antimicrobial against gram negative bacteria.

It is also contemplated that the composition of the present disclosure may be effective as a spermicidal and antimicrobial agent, which could help prevent the spread of sexually transmitted diseases. Prevention of HPV transmission, along with vaccines, may markedly reduce cervical cancer, as well as some vulvar, penile, anal/rectal, and oral pharyngeal carcinomas. The composition of the present disclosure may be administered alone or in combination with one or more barrier methods of contraception, such as a diaphragm, sponge, male condom, female condom, etc. It is contemplated that the composition could be effective in the prevention of transmission of sexually transmitted diseases.

The composition of the present disclosure may be utilized as a cleaner/antiseptic and may be used on living and non-living surfaces. The composition of the present disclosure may provide a means by which biofilms as formed by bacterial and fungal organisms are disrupted and the embedded organisms in the biofilm, whether they are biofilm formers or present by accidental inclusion, entrapment or otherwise, are killed or rendered non-viable and/or otherwise non-threatening. The composition of the present disclosure may provide a means by which bacterial and yeast spores are either killed outright in the spore stage, or are rendered ineffective by being unable to germinate, or by germination followed by rapid killing before their pathogenic potential is expressed. The composition of the present disclosure may provide a means by which quorum sensing (QS) mechanisms used by biofilm forming microorganisms is interrupted. While these mechanisms vary from organism to organism, and gram-positive organisms use a somewhat different QS system from gram-negative, in all cases there is a molecule or series of molecules which provide the QS function. In many cases, these are protein molecules that are potentially susceptible to structural changes by reaction with the composition of the present disclosure. Reactions such as hydrolysis, alcoholysis, esterification, transesterification, oxidation, protein denaturation, or chelation of both free ions and partially bound cations are likely possibilities. Substances which are targets for hydrolytic or alcoholytic destruction or disruption are, for example, acylhomoserine lactones (AHL's, the QS molecules of gram-positive biofilm formers), and other lactone or ester components of gram-positive bacteria. Biofilms are also known to be disrupted by chelators, which it is believed, results from the binding effect of the chelator on divalent, trivalent, or other cations necessary for the formation of the biofilm. Inclusion of one or more chelators, including but not limited to EDTA, provides this function to the composition of the present disclosure.

The composition of the present disclosure may provide a means by which biofilms as formed by bacterial and fungal organisms are disrupted and the embedded organisms in the biofilm, whether they are biofilm formers or present by accidental inclusion, entrapment or otherwise, are killed or rendered non-viable and/or otherwise non-threatening.

In embodiments in which the composition is used on non-living surfaces, in addition to providing disinfection at the time of the application, the composition may also provide a lasting residual barrier against contamination. For example, even after volatile constituents of the composition (e.g., water, alcohol, hydrogen peroxide, etc.) have evaporated, the chelating agent and/or the procoagulant agent may remain on the treated surfaces (e.g., multiple use vial or port cleaning/protecting device, stethoscope, fingers, other tissue, etc.) as a barrier that will provide antibacterial, antifungal or sporicidal (e.g., preventing germination of the spores), anti-parasitic, spermicidal or spermiostatic (e.g., decrease the motility of spermatozoon) and antiviral qualities. For example, by robbing the environment of components (e.g., iron, magnesium, and manganese) that are needed for the bacteria, spores, parasites, fungus, and viruses to reproduce, the chelating agent provides a lasting defense to contamination even after other constituents of the composition have evaporated.

In embodiments, the composition may be applied to surgical gloves and/or surgical instruments. Surgical site infections (and infections to surgeons, nurses, etc.) may result from inadequate antiseptic preparations of the skin and surgical instruments. Gloves can get punctured, and if sharp debridement is used, then punctures are more likely. An alcohol surgical rub of the hands, after it dries off, loses its antibacterial effect.

In embodiments, the composition may be applied to surfaces to inactivate pathogens or toxins on the surfaces. For example, the composition may be applied to animate surfaces (e.g., skin, mucous membranes, etc.) and inanimate surfaces (e.g., clothing, face masks, weapons, implements, equipment etc.) exposed to biological agents.

In embodiments, the composition can be used as a pre-exposure treatment for skin, mucous membranes, and/or clothing materials. In embodiments, the composition can be used post-exposure to decontaminate military personnel and/or civilians exposed to biological pathogens or toxins as bioweapons (e.g., Bacillus anthracis spores, Clostridium tetani spores or toxins, Staphylococcus aureus toxins, Ebola virus, respiratory viruses, etc.) A nontoxic irrigant, wash, spray, aerosol, slurry, liquid (e.g., via soaking), etc. could be used to apply the composition to the surfaces.

The composition may be prepared in bulk for mass casualties, and in embodiments, the composition may be prepared in advance. Some or all of the water in the composition may be added on-site such as in a certain the location where the casualties occurred. If soldiers or civilians have abraded skin due to missile, blast, thermal or radiological burn injuries, the immunomodulatory, hemostatic, and tissue protective and regenerative effects of the composition would be beneficial.

In an embodiment, it is contemplated that the composition described herein may be used inside of a surgical glove to provide additional antimicrobial protection and procoagulant activity in the event of microscopic puncture of the glove. In an embodiment, the composition described herein is combined with a lubricant in a glove to be worn by a medical professional.

If bacteria, viruses, fungi, etc. remain on their hand after washing, and a puncture in a glove is present or develops in the glove worn on that hand, the composition's antimicrobial activity would kill the bacteria, viruses, fungi, etc. on a surgeon's or nurse's hand before it is spread to the patient.

Similarly, if bacteria, viruses, fungi, etc. is outside of a glove with a puncture (e.g., in a patient's blood) but the glove is coated inside with the composition described herein, the medical professional may be protected by the combined antimicrobial and procoagulant activity of the composition.

Whether applied to surgical gloves or surgical instruments, the composition may, in addition to providing disinfection at the time of the application, provide a lasting barrier against contamination. For example, even after volatile constituents of the composition (e.g., water, alcohol, hydrogen peroxide, etc.) have evaporated, the chelating agent and/or the procoagulant agent may remain on the treated surfaces (e.g., multiple use vial or port cleaning/protecting device, stethoscope, fingers, other tissue, etc.) as a barrier that will provide antibacterial, antifungal or sporicidal (e.g., preventing germination of the spores), anti-parasitic, spermicidal or spermiostatic (e.g., decrease the motility of spermatozoon) and antiviral qualities. EDTA and the procoagulant agent in the composition described herein leave a residual antimicrobial in effect. The composition leaves EDTA, chitosan and/or polyphosphate, to prevent bacterial, fungal, and likely viral, colonization. For example, by robbing the environment of components (e.g., iron, magnesium, and manganese) that are needed for the bacteria, spores, parasites, fungus, and viruses to reproduce, the chelating agent provides a lasting defense to contamination even after other constituents of the composition have evaporated.

### Illustrative Method of Use

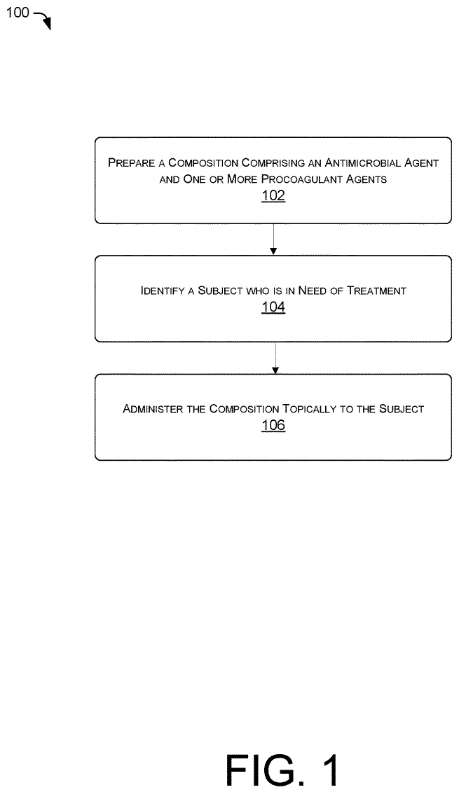

FIG. 1 is a flow diagram of an embodiment of a method of use 100 of the composition as described above. In particular, FIG. 1 illustrates an embodiment in which the method of use comprises topically applying delivering the composition, to a subject in need thereof.

At step 102, a composition is prepared that contains at least one antimicrobial agent as described herein and at least one procoagulant agent as described herein. Optional additional ingredients may also be added. The composition may be in solution, or may be in other forms, such as gel, cream, ointment, drops, spray, slurry, etc.

At step 104, a subject may be identified who is in need of treatment. In examples, the subject may have an open wound.

At step 106, the composition is administered to the subject. In embodiments, the composition may be sprayed on the wound, may be applied to the wound via a wound dressing, may be applied as a hydrogel or liquid, etc.

### Illustrative Method of Manufacture

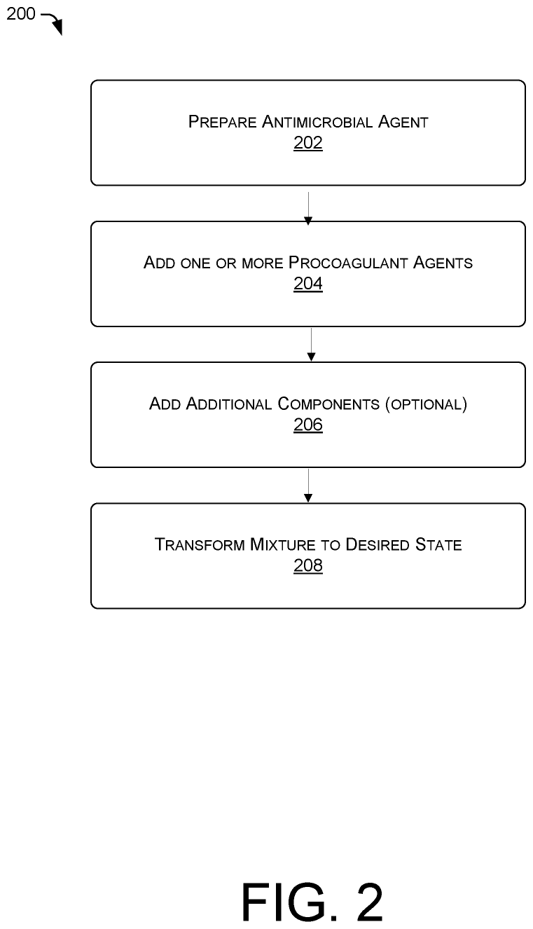

FIG. 2 is a flow diagram of an embodiment of a process of manufacture 200 of the composition.

As described above, the composition described herein includes at least one antimicrobial agent and at least one procoagulant agent and may contain optional additional components.

At step 202, the components of the antimicrobial formulation(s) are combined. As described above, an antimicrobial formulation may include, but is not limited to, at least,
alcohol from about 1% to about 85% w/w, or from about 20% to about 60% w/w, or about 50% w/w;
peroxide or peroxide generating agent from about 0.05% to about 20% w/w, from about 0.05% to about 10% w/w, or about 1.5% w/w;
EDTA from about 0.5 mg/mL to about 5 mg/mL, from about 0.4 mg to about 10 mg/mL, from about 0.3 mg to about 15 mg/mL, or about 10 mg/mL;
procoagulant from about 1% to about 95%; and
the balance of water.

One or more components may be added to the antimicrobial agent later than the others. In an example, a component may be added to the other components immediately before use. The composition may be manufactured as one or more of a powder, a gel, an emulsion, or a liquid.

At step 204, at least one procoagulant agent may be added to the at least one antimicrobial agent, as described above.

At step 206, optional additional components may be added. Optional additional components may include, but are not limited to, an acidic agent, a viscosity-increasing agent, host MMPs, TNF-alpha, bacterial beta-lactamases, (including multi-resistant extended spectrum beta-lactamases (ESBL)), carbapenemases, and metallocarbamases, etc.

At step 208, the composition may be transformed into a desired state if not already in the desired state. For example, a composition in the form of a liquid may be transformed to a gel to prepare it for application.

### Clauses

Clause 1: A composition comprising:

an antimicrobial agent comprising:
(a) water;
(b) a low molecular weight alcohol;
(c) a peroxide or peroxide-generating agent; and
(d) a chelating agent; and

one or more procoagulant agents.

Clause 2: The composition of clause 1, wherein the one or more procoagulant agents comprises polyphosphate.

Clause 3: The composition of clause 2, wherein the polyphosphate is present in the composition at a non-toxic concentration of at least one of from about 1% to about 20% weight to weight (w/w), or from about 5% to about 15% w/w, or about 10% w/w.

Clause 4: The composition of any of clauses 1-3, wherein the one or more procoagulant agents includes chitosan, polyphosphate, collagen, hyaluronan, high molecular weight hyaluronic acid, kaolin or any combination thereof.

Clause 5: The composition of clause 4, wherein the chitosan is present in the composition at a non-toxic concentration of at least one of from about 1% to about 95% w/w, or from about 20% to about 60% w/w, or about 50% w/w.

Clause 6: The composition of any of clauses 1-5, wherein the alcohol comprises ethanol and is present in the composition of at least one of from about 1% to about 85% w/w, or from about 10% to about 70% w/w, or about 50% w/w.

Clause 7: The composition of any of clauses 1-6, wherein the peroxide or peroxide-generating agent comprises hydrogen peroxide (H₂O₂) and is present in the composition at a concentration of at least one of from about 0.05% to about 20% w/w, from about 0.05% to about 10% w/w, or about 1.5% w/w.

Clause 8: The composition of any of clauses 1-7, wherein the chelating agent comprises ethylenediamine tetraacetic acid (EDTA), acids of EDTA, salts of EDTA, or any combination thereof and is present in the composition at a concentration of at least one of from about 0.5 mg/mL to about 5 mg/mL, from about 0.4 mg to about 10 mg/mL, from about 0.3 mg to about 25 mg/mL, or about 10 mg/mL.

Clause 9: The composition of any of clauses 1-8, further comprising a viscosity-increasing agent.

Clause 10: The composition of any of clause 1-9, wherein the viscosity-increasing agent comprises hydroxypropyl methylcellulose (HPMC).

Clause 11: The composition of any of clauses 1-10, wherein the composition comprises two or more procoagulant agents.

Clause 12: The composition of any of clauses 1-11, wherein the procoagulant agent comprises collagen.

Clause 13: The composition of clause 12, wherein the collagen is present in the composition at a non-toxic concentration of at least one of from about 1% to about 95% w/w, or from about 20% to about 60% w/w, or about 50% w/w.

Clause 14: The composition of any of clauses 1-13, wherein one or more components of the antimicrobial agent and one or more components of the procoagulant agent are covalently linked.

Clause 15. The composition of any of clauses 1-14, wherein the one or more procoagulant agents comprises hyaluronan or hyaluronic acid.

Clause 16. The composition of clause 15, wherein the hyaluronan or hyaluronic acid is present in the composition at a non-toxic concentration of at least one of from about 1% to about 95% w/w, or from about 20% to about 60% w/w, or about 50% w/w.

Clause 17. The composition of any of clauses 1-16, wherein the one or more procoagulant agents comprises kaolin.

Clause 18. The composition of clause 17, wherein the kaolin is present in the composition at a non-toxic concentration of at least one of from about 1% to about 95% w/w, or from about 20% to about 60% w/w, or about 50% w/w.

Clause 19. A method of method of treating, inhibiting, or reducing microbial growth or infection at a wound site and encouraging coagulant activity at the wound site, comprising:

identifying a site of the wound; and

applying the composition of clause 1 to the site.

Clause 20. A method of treating, inhibiting, or reducing a condition or disease, comprising administering to a subject a therapeutically effective amount of composition comprising:

(a) water;

(b) a low molecular weight alcohol;

(c) a peroxide or peroxide-generating agent;

(d) a chelating agent; and

(e) one or more procoagulant agents.

Clause 21. The method of clause 20, wherein the condition or disease is at least one of bacterial condition or disease, a fungal condition or disease, a viral condition or disease, a parasitic condition or disease, a biological toxin disease or condition, an inflammatory condition or disease, immune response condition or disease, or a pre-cancerous or cancerous condition.

Clause 22. The method of any of clause 20 or 21, wherein the condition or disease is bleeding.

Clause 23. The method of any of clauses 20-22, wherein the composition is administered by at least one of topical application; intravenous injection; intraperitoneal injection or implantation; intramuscular injection or implantation; intralesional injection; subcutaneous injection or implantation; intradermal injection; suppositories; tampons; pessaries; enteric application; or nasal route.

Clause 24. The method of any of clauses 20-23, wherein the composition is applied to a site comprising one or more of a wound site, a catheter site, a surgical site, an injection site, a catheter, a catheter lumen, a vulvar carcinoma site, a penile carcinoma site, an anal/rectal carcinoma site, a thermal burn site, a chemical burn site, a radiation burn site, a skin lesion, oral sites, bony sites, cartilage sites, joint sites, anal sites, vaginal sites, cervical sites, vulvar sites, penile sites, ulcerated skin sites, acne sites, actinic keratosis sites, inflamed sites, irritated sites, gastric sites, gastrointestinal sites, esophageal sites, esophagogastrointestinal sites, intestinal sites, cardiac sites, vascular sites, nasal sites, nasopharyngeal sites, or aural sites.

Clause 25. The method of any of clauses 20-24, wherein:

the alcohol comprises ethanol;

the chelating agent comprises ethylenediamine tetraacetic acid (EDTA), acids of EDTA, salts of EDTA, or any combination thereof;

the peroxide or peroxide-generating agent comprises hydrogen peroxide (H₂O₂); and

the one or more procoagulant agents comprises chitosan.

Clause 26. The method of any of clauses 20-24, wherein:

the alcohol comprises ethanol;

the chelating agent comprises ethylenediamine tetraacetic acid (EDTA), acids of EDTA, salts of EDTA, or any combination thereof;

the peroxide or peroxide-generating agent comprises hydrogen peroxide (H₂O₂); and

the one or more procoagulant agents comprises kaolin.

Clause 27. The method of any of clauses 20-24, wherein:

the alcohol comprises ethanol;

the chelating agent comprises ethylenediamine tetraacetic acid (EDTA), acids of EDTA, salts of EDTA, or any combination thereof;

the peroxide or peroxide-generating agent comprises hydrogen peroxide (H₂O₂); and

the one or more procoagulant agents comprises hyaluronan.

Clause 28. A method of manufacturing a composition comprising at least:

combining components comprising:
(a) water;
(b) a low molecular weight alcohol;
(c) a peroxide or peroxide-generating agent;
(d) a chelating agent; and
(e) one or more procoagulant agents; and

putting the components in solution.

Clause 29. A wound dressing comprising:

a lattice of at least one of fibers or nanoparticles;

a composition applied to the lattice comprising:
(a) water;
(b) a low molecular weight alcohol;
(c) a peroxide or peroxide-generating agent;
(d) a chelating agent; and
(e) one or more procoagulant agents.

### CONCLUSION

Although the subject matter has been described in language specific to structural features and/or methodological acts, it is to be understood that the subject matter defined in the appended claims is not necessarily limited to the specific features or acts described. Rather, the specific features and acts are disclosed as illustrative forms of implementing the claims.

One skilled in the art will realize that a virtually unlimited number of variations to the above descriptions are possible, and that the examples and the accompanying figures are merely to illustrate one or more embodiments or examples of implementations.

It will be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter not be limited to the particular embodiments disclosed, but that such claimed subject matter may also include all embodiments falling within the scope of the appended claims, and equivalents thereof.

In the detailed description above, numerous specific details are set forth to provide a thorough understanding of claimed subject matter. However, it will be understood by those skilled in the art that claimed subject matter may be practiced without these specific details. In other instances, methods, apparatuses, or systems that would be known by one of ordinary skill have not been described in detail so as not to obscure claimed subject matter.

Reference throughout this disclosure to “one embodiment” or “an embodiment” may mean that a particular feature, structure, or characteristic described in connection with a particular embodiment may be included in at least one embodiment of claimed subject matter. Thus, appearances of the phrase “in one embodiment” or “an embodiment” in various places throughout this specification are not necessarily intended to refer to the same embodiment or to any one particular embodiment described. Furthermore, it is to be understood that particular features, structures, or characteristics described may be combined in various ways in one or more embodiments. In general, of course, these and other issues may vary with the particular context of usage. Therefore, the particular context of the description or the usage of these terms may provide helpful guidance regarding inferences to be drawn for that context.

Conditional language such as, among others, “can,” “could,” “might” or “may,” unless specifically stated otherwise, are understood within the context to present that certain examples or embodiments include, while other examples or embodiments do not include, certain features, elements and/or steps. Thus, such conditional language is not generally intended to imply that certain features, elements and/or steps are in any way required for one or more examples or embodiments or that one or more examples or embodiments necessarily include logic for deciding, with or without user input or prompting, whether certain features, elements and/or steps are included or are to be performed in any particular example or embodiment. Conjunctive language such as the phrase “at least one of X, Y or Z,” unless specifically stated otherwise, is to be understood to present that an item, term, etc., can be either X, Y, or Z, or a combination thereof

We claim:

1. A composition comprising:
an antimicrobial agent comprising:
(a) water;
(b) a low molecular weight alcohol;
(c) a peroxide or peroxide-generating agent; and
(d) a chelating agent; and
one or more procoagulant agents.

2. The composition of claim 1, wherein the one or more procoagulant agents comprises polyphosphate.

3. The composition of claim 2, wherein the polyphosphate is present in the composition at a non-toxic concentration of at least one of from about 1% to about 20% weight to weight (w/w), or from about 5% to about 15% w/w, or about 10% w/w.

4. The composition of claim 1, wherein the one or more procoagulant agents includes chitosan, polyphosphate, collagen, hyaluronan, high molecular weight hyaluronic acid, kaolin or any combination thereof.

5. The composition of claim 4, wherein the chitosan is present in the composition at a non-toxic concentration of at least one of from about 1% to about 95% w/w, or from about 20% to about 60% w/w, or about 50% w/w.

6. The composition of claim 1, wherein the alcohol comprises ethanol and is present in the composition of at least one of from about 1% to about 85% w/w, or from about 10% to about 70% w/w, or about 50% w/w.

7. The composition of claim 1, wherein the peroxide or peroxide-generating agent comprises hydrogen peroxide (H₂O₂) and is present in the composition at a concentration of at least one of from about 0.05% to about 20% w/w, from about 0.05% to about 10% w/w, or about 1.5% w/w.

8. The composition of claim 1, wherein the chelating agent comprises ethylenediamine tetraacetic acid (EDTA), acids of EDTA, salts of EDTA, or any combination thereof and is present in the composition at a concentration of at least one of from about 0.5 mg/mL to about 5 mg/mL, from about 0.4 mg to about 10 mg/mL, from about 0.3 mg to about 25 mg/mL, or about 10 mg/mL.

9. The composition of claim 1, further comprising a viscosity-increasing agent.

10. The composition of claim 1, wherein the viscosity-increasing agent comprises hydroxypropyl methylcellulose (HPMC).

11. The composition of claim 1, wherein the composition comprises two or more procoagulant agents.

12. The composition of claim 1, wherein the procoagulant agent comprises collagen.

13. The composition of claim 12, wherein the collagen is present in the composition at a non-toxic concentration of at least one of from about 1% to about 95% w/w, or from about 20% to about 60% w/w, or about 50% w/w.

14. The composition of claim 1, wherein one or more components of the antimicrobial agent and one or more components of the procoagulant agent are covalently linked.

15. The composition of claim 1, wherein the one or more procoagulant agents comprises hyaluronan or hyaluronic acid.

16. The composition of claim 15, wherein the hyaluronan or hyaluronic acid is present in the composition at a non-toxic concentration of at least one of from about 1% to about 95% w/w, or from about 20% to about 60% w/w, or about 50% w/w.

17. The composition of claim 1, wherein the one or more procoagulant agents comprises kaolin.

18. The composition of claim 17, wherein the kaolin is present in the composition at a non-toxic concentration of at least one of from about 1% to about 95% w/w, or from about 20% to about 60% w/w, or about 50% w/w.

19. A method of method of treating, inhibiting, or reducing microbial growth or infection at a wound site and encouraging coagulant activity at the wound site, comprising:
identifying a site of the wound; and
applying the composition of claim 1 to the site.

20.-28. (canceled)

29. A wound dressing comprising:
a lattice of at least one of fibers or nanoparticles;
a composition applied to the lattice comprising:
(a) water;
(b) a low molecular weight alcohol;
(c) a peroxide or peroxide-generating agent;
(d) a chelating agent; and
(e) one or more procoagulant agents.
